(19) United States
(12) Patent Application Publication
(10) Pub. No.: US 2009/0048081 A1
(43) Pub. Date: Feb. 19, 2009

(54) ORTHOPEDIC THERAPY SYSTEM AND DEVICE AND A METHOD OF USE

(76) Inventor: Paul KAMINS, Lancaster, NH (US)

Correspondence Address: PERRY E. VAN OVER & ASSOCIATES, PLLC, P.O. BOX 399, FAIRFAX, VA, 22038, US

(21) Appl. No.: 11/840,712

(22) Filed: Aug. 17, 2007

(51) Int. Cl. A63B 21/00; A61B 5/103; A61H 1/00

(52) U.S. Cl. 482/131; 600/595; 601/5

(57) ABSTRACT

Provided is a novel physical therapy system and device that can be manually operated by a post-operative patient without the need of professional on site assistance, wherein the degree of movement of the limb or joint of interest is measured and recorded so as to provide a record of the progress of the prescribed post-operative therapy for immediate feedback to the patient as well as for long term data recordation for the therapist or physician.

### BACKGROUND OF THE INVENTION

1. Technical Field

The present invention relates to orthopedic therapy and in particular to devices that provide dynamic measurable therapy for use by orthopedic patients. More particularly, the present invention relates to a physical therapy system and device that can be manually operated by a post-operative patient without the need of professional on site assistance, wherein the degree of movement of the limb or joint of interest is measured and recorded so as to provide a record of the progress of the prescribed post-operative therapy for immediate feedback to the patient as well as for long term data recordation for the therapist or physician.

2. Background Art

The success of joint reconstructive or replacement surgery depends largely upon the postoperative rehabilitation process. This is particularly true for a total knee arthroplasty. In total knee arthroplasty surgeons are typically able to achieve a great range of joint motion for the sedated patient on the operating table. However, the quantity and quality of the post-operative therapy is critical to the patient's ultimate range of motion and therefore, the ultimate success of the surgical procedure. A limited range of motion of a post-operative joint, particularly a knee, will result in pain and limited function. It is generally understood that there is a six-week window of opportunity after surgery during which a patient can, through therapy and exercise, maximize his ultimate range of motion for the post-operative joint. Very important to the success of any post-operative therapy is the patient's commitment to maximizing the effort to rehabilitate the affected joint. Conventional post-operative therapies necessarily have to enlist the patient's commitment to and compliance with the prescribed regime as much as possible. However, the success of the postoperative therapy in large part relies more on the therapist contribution than on passive mechanized therapy devices that may or may not be properly, routinely, and adequately employed by the patient.

Continuous passive motion (CPM) machines and/or manually applied physical therapy to an injured or post operative limb are the primary rehabilitation treatments chosen by most doctors and therapists. Many limb and joint exercising devices are known. Generally, these machines have a motor driven limb support with the limb support capable of being set to periodically move the limb in a preset range of positions for a preset length of time and at a preset speed. Once these parameters have been selected the machine automatically moves the limb from a straightened position back and forth to these pre-selected positions at the pre-selected speeds and durations.

One major drawback to these motor driven machines is the problem of selecting the optimum settings for the machine. While resetting positions and safety cut-off switches are usually provided for the user, the initial settings for the machines, which are set by the therapist, are frequently too severe and painful. Later, as the therapy progresses and the joint becomes more limber and capable of greater movement the settings are often insufficient and therefore less helpful in taking the rehabilitation as far as possible.

Further, conventional therapy machines and therapists cannot sense the level of pain experienced by the patient during movement of the joint or the true degree of stiffness of the joint. For this reason, the rehabilitative therapy provided to the patient may be too severe or too reserve. Only the patient is truly aware of what joint motion limits he is capable of reaching on any given day of the rehabilitative period.

A conventional rehabilitative therapy regime for a post-operative total knee replacement would include both the use of a therapist and a continuous passive motion (CPM) machine. In use, the patient would lie down and securely strap his knee into the machine. The machine with its preset motion, speed, and duration parameters would be turned on while the patient passively endured the machine bending the knee to a prescribed range of motion. The range of motion can be adjusted by the therapist to a level he believes to be within the patient's pain and motion tolerance; however the level of uncertainty of the optimum settings for any given patient is high. The CPM is typically prescribed for self-use at home by the patient for a period of several weeks after surgery. During this rehabilitative period the involvement of a physical therapist in providing manipulative therapy and in monitoring the use of the CPM is generally considered the most important element in a successful rehabilitation regime. Even if the settings on the CPM are relatively appropriate for the patient, it remains that the effectiveness of the CPM in the rehabilitative regime is totally dependent upon the amount of effective use it receives. The physical therapist working with the postoperative patient can improve the rehabilitation progress by monitoring the patient's use of the CPM and by passively stretching the patient's knee and recording the progress; however this spot-checking of the patient's use of the CPM is at best periodic and isolated and not necessarily a true picture of whether or not the patient is making good use of the machine. Further, inconvenience, forgetfulness, pain avoidance and many other reasons can contribute to the patient's neglect of his prescribed CPM and home therapy regime. For this reason, the therapist's sessions with the patient are often the most effective part of the rehabilitative effort. Unfortunately, the patient's sessions with the therapist will, at best, be limited to about 45 minutes for each of two or three sessions per week for six weeks. Over the six week long post-operative period the therapist will likely spend no more than 13 to 15 hours working with the patient. For this reason, a good therapist will typically prescribe a variety of additional exercises to assist the patient in the effort of stretching the knee into flexion and extension during those long periods when the patient is at home without the benefit of the therapist presence. For example, extension of the knee joint is often augmented by having the patient prop an object under his heel after which the patient will press the knee downward from above. Flexion exercises are more difficult. Efforts to improve flexion of the knee can be augmented by the patient sitting on a chair and pulling the operative leg back with the opposite leg. While these and other self-help exercises can be useful, they are only beneficial if the patient exercises a great deal of self-discipline and strictly adheres to the prescribed program. The patient is typically left alone to exercise or to neglect his prescribed therapy program for the majority of those initial few weeks when a therapy program can be most effective. Further, although the time during which the therapist is actively involved in sessions with the patient is of great value, it remains that providing such professional assistance is becoming increasingly expensive for the patient and his insurance company. Limited finances or insurance can curtail or abruptly end the active assistance of a professional therapist. For this reason, it is very important that, within reasonable cost constraints, the best possible tools be provided to aid the patient in the self-administered portion of the exercise program.

Another important aspect of the patient's rehabilitation program is the requirement to accurately measure and report the degree of flexion and extension of the joint in a timely manner. It is very important that the therapist and the surgeon are aware of the progress being made by the patient in order to permit course corrections in the therapy program as needed. At present, the degree of flexion or extension of a joint is measured by the therapist using a goniometer, an angle finder, which the therapist holds on the side of the patient's knee and uses his best-guess to align the device with the leg. The therapist generally uses this conventional angle measurement technique intermittently to determine the level of progress in the rehabilitative effort being made by the patient. Studies presented at the Orthopedic Trauma Association (OTA) 2000 meeting indicated that a visual inspection and angle estimation of a joint by an attending surgeon, an orthopedic fellow, and an orthopedic senior resident varied from the actual angle of the joint as determined by radiological examination by an average of 6.5° and the difference between the goniometer measurement and the radiologically determined angle was only slightly better at 5.60. In addition, a significant difference in 1 of 8 comparisons among attending surgeons, fellows and residents was found (Poster No. 82, OTA 2000 Posters). Further, the inaccuracy of visual or goniometer estimates of a joint angle can also be attributable to the obvious possibility that the therapists estimate of proper alignment of the goniometer to the patient's leg can vary considerably from one visit to the next; what seems like progress from an earlier session may only be the result of inaccurate instrument alignment at a subsequent session. This is particularly true when a substitute therapist must attend to the patient. In such cases an inconsistent alignment of the goniometer should be expected and therefore the recorded progress would be expected to be erroneous.

To date there has been no therapy system provided that effectively enables the patient to completely control all parameters of his rehabilitative machine exercise therapy so as to actively stretch the knee joint with immediate biofeedback as well as precise recordation of therapy progress. Further, the accuracy of any recordation of the level of progress using conventional measurement techniques and devices is at best questionable. What is needed is a joint rehabilitative therapy system that allows the patient alone to conveniently use the device on a daily basis wherein the parameters of the degree of extension and flexion, speed, and duration are established by the patient using a real time biofeedback mechanism and allowing the patient to limit or extend the effort to conform to his pain threshold rather to that which a therapist has applied a best-guess.

### SUMMARY OF THE DISCLOSURE°

It is a primary object of the present invention to meet the above identified need by providing a novel exercise therapy system for rehabilitation of a post-operative joint that can be manually operated by the patient, the system being capable of providing both immediate biofeedback to the patient and recordation of the therapy progress.

It is another object of the invention to provide a system for rehabilitative exercise therapy that is convenient and safe to be used under the manual control of the patient such that the patient, being fully aware of the pain level and joint movement limitations, can maximize the benefit of the therapy in a controlled, measured manner with the benefit of immediate biofeedback on the degree of extension and flexion of the affected joint.

It is another object of the invention to provide a system for rehabilitative exercise therapy that the patient can easily adjust to increase the therapeutic benefit, without the need of professional assistance, as the course of rehabilitative therapy progresses.

It is another object of the present invention to provide a system for rehabilitative exercise therapy that can be ergonomically configured to provide a stable platform to maximize biomechanical leverage for exercise therapy that is controlled and adjusted by the patient using information provided by the immediate biofeedback of degree of joint extension and flexion achieved.

It is another object of the invention to provide a system for joint rehabilitation exercise therapy that records the progress of the therapy for study by the surgeon or therapist.

It is another object of the invention to provide a system for joint rehabilitation that is operated by the patient alone, the system including a remote data link capable of transmitting printable information on the progress of the therapy to a surgeon or therapist supervising the therapy progress.

It is another object of the invention to provide a system for joint rehabilitation that includes an exercise platform having mechanisms with servo-assist that are capable of ergonomically reconfiguring the platform as needed.

Also provided is a novel system for joint rehabilitation therapy that can be provided as a kit to be easily assembled and operated by a patient without the need for professional assistance.

Also provided is a method for performing effective joint rehabilitation therapy using the system of the present invention.

### BRIEF DESCRIPTION OF THE DRAWINGS

The foregoing and other features of the disclosed device will become apparent to one skilled in the art to which the present invention relates upon consideration of the following description of exemplary embodiments with reference to the accompanying drawings, wherein:

FIG. 1 shows an isometric view of the novel therapy system including the ergonomically adjustable device frame for supporting the patient during the exercise regime, the biofeedback assembly, and the data recordation assembly.

FIG. 2 shows a side view of the novel therapy system with an exemplary device frame.

FIG. 3 shows a top view of the novel therapy system with an ergonomically configured sidewall taper.

FIG. 4 shows an isometric view of the novel therapy system including another example of the platform assembly with an alternative step adjustment mechanism.

FIGS. 5A-5B respectively show some components of the device frame of FIG. 4 and a detail of the alternative step adjustment mechanism and method of step adjustment.

FIG. 6 shows depictions of a patient's leg in flexion, extension, and partial extension positions with the distal end of a cable extending under coiled tension from a linear cable encoder properly positioned and attached on the lateral aspect of the patient's leg (to the outer garment in practice) during use of the system. Exemplary comparative measurements of the cable length for each of the three positions are annotated on the figure.

FIG. 7 shows a diagram of an exemplary joint angle measurement mechanism including a depiction of a the lateral aspect of a patient's leg with a tension coiled cable extending from a linear cable encoder properly attached (to the outer garment in practice) and an exemplary display showing the upper leg and lower leg measured preset lengths of “A” and “B” respectively, the measured distance of “L” as determined by the length of the cable of a linear cable encoder, the programmed algorithm of the linear cable encoder computing device, and an exemplary display of the present parameters, measured cable length, computed angle “C” of the joint at issue, and a display of real time computations of flexion and extension as well as a display of flexion and extension progress over time as can be recorded in the computing device of the encoder.

FIG. 8 shows an isometric view of the novel therapy system including an alternative embodiment of the platform assembly. The exemplary alternative platform assembly can be of uni-body construction and can have servo-assisted upward telescoping left and right side assemblies as well as servo-assisted step and handle position adjustments. The system can also be equipped with a variety of alternative biofeedback assemblies and be suitably employed in a high use environment such as a therapy department or the office of a therapist.

FIG. 9 shows an isometric view of the novel therapy system including an alternative single upright support embodiment of the platform assembly.

FIGS. 10A-b respectively show (FIG. A) a side view of the novel therapy system with an exemplary device frame with a depiction of a patient physically positioned on the exercise platform with the cable clip of the linear cable encoder properly positioned adjacent to the patient's hip joint and attached to the patient's outer garment; (FIG. B) a close-up view of the cable clip showing the preferred embodiment of a pin, which can be attached to the patient's clothing and securing a two-part quick disconnect with an attached cable of a linear cable encoder.

FIG. 11 shows some representative components of the novel therapy system and a transport container for the same that can be provided as part of a kit. The device so provided can be easily assembled and disassembled for return shipment to the provider.

### DETAILED DESCRIPTION OF PREFERRED EMBODIMENTS

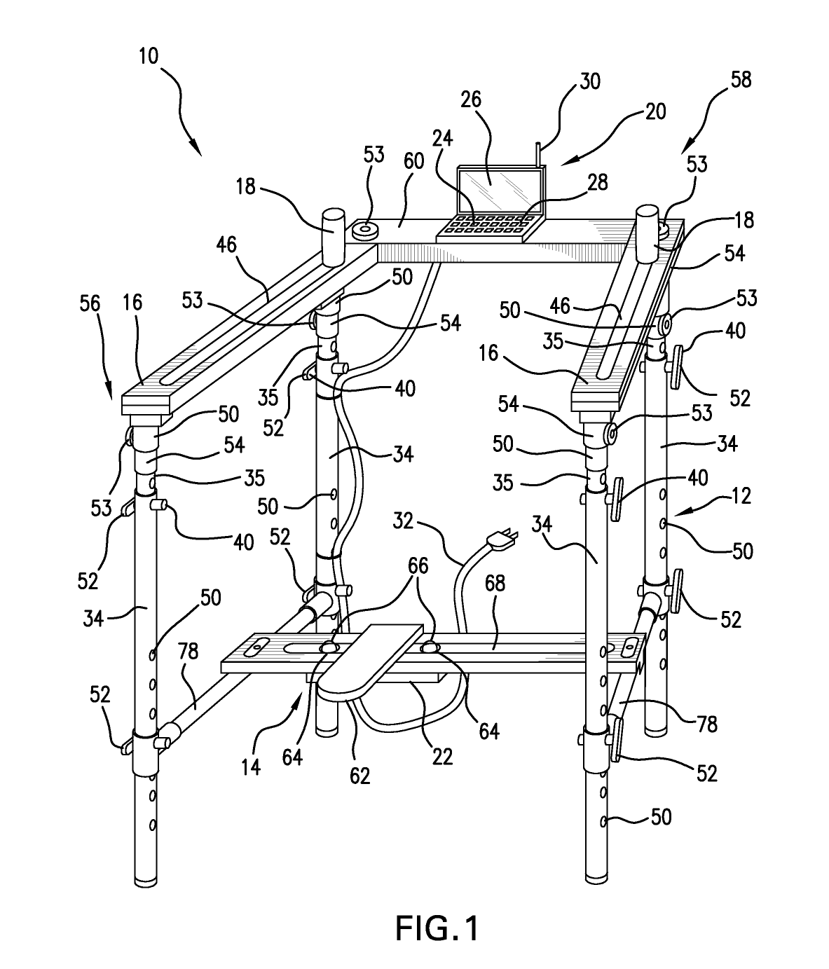

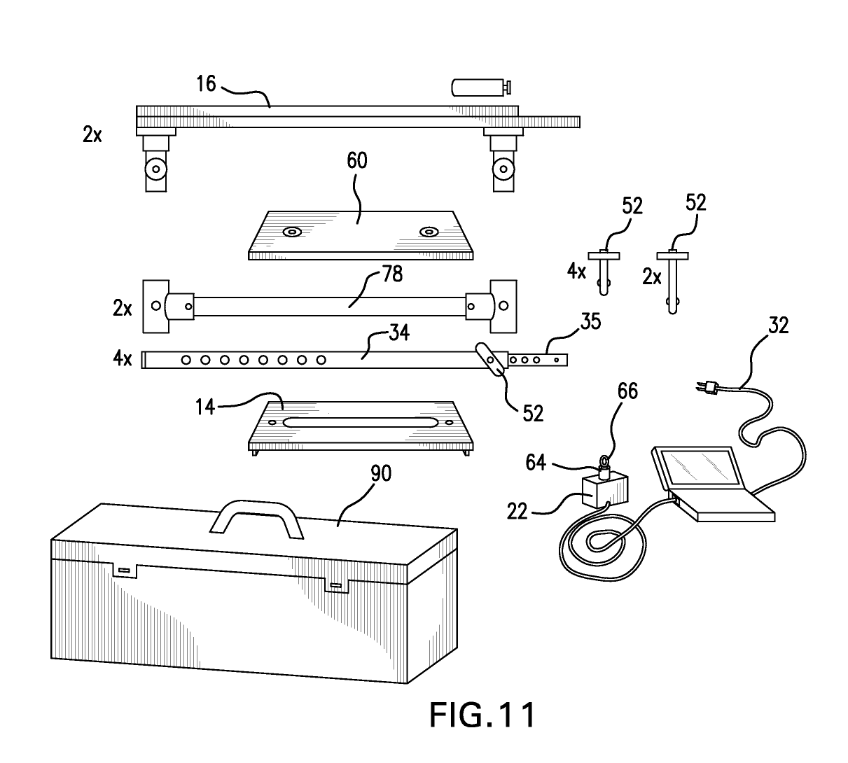

Detailed embodiments of the present invention are disclosed herein and represented in FIGS. 1-11; however, it is understood that the following description and each of the accompanying figures are provided as being exemplary of the invention, which may be embodied in various forms without departing from the scope of the claimed invention. Thus, the specific structural and functional details provided in the following description are non-limiting, but serve merely as a basis for the invention as defined by the claims provided herewith. The system described below can be modified as needed to conform to further development and improvement of materials without departing from the inventor's concept of the invention as claimed.

The concept of the invention is to provide a system that can be used by a patient for exercise therapy for any part of the body requiring post-operative or post-injury physical rehabilitation. The following non-limiting description is directed to the invention configured for use by a patient requiring therapy for the knee joint; although the same principles of the invention can be adapted to provide exercise therapy for other joints such as, for example, shoulder, elbow, wrist, hip, ankle, and hand.

The present invention, in contrast to the conventional continuous passive machine (CPM), which uses a motorized mechanism to move the affected joint of the patient, can be viewed as a continuous active machine (CAM), in which the patient takes an active role in moving the affected joint as well as in controlling the steady progress of the therapy regime. The present invention enables the patient to continually use the system on a daily basis as compared to conventional therapy programs, which include only isolated visits with the therapist.

It is of great advantage that the present invention is a system that can be shipped disassembled to a patient's home and then assembled for use in a convenient, frequently accessed area where the patient without any professional assistance can use the invention throughout the day to achieve a continuing active therapy regime that provides immediate feedback to the patient on the level of progress achieved. Unlike the conventional CPM device, the present invention allows the patient to monitor the progress of the therapy and conveniently take an active roll in the daily progress of the therapy. The real time feedback of improvement or lack thereof in the angle or range of joint articulation by the patient enables the patient to immediately identify his daily progress, challenge himself, and adjust his exercise regime to maximize the benefit of the system.

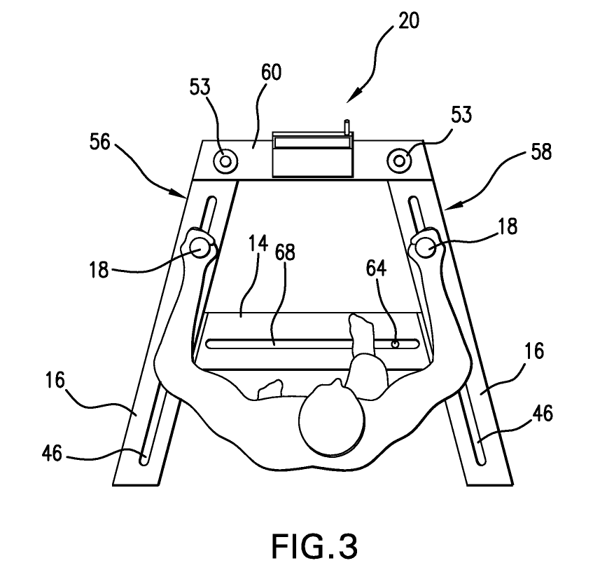

The exemplary orthopedic therapy system described herein and generally shown in the accompanying figures at 10 includes an exercise platform assembly, generally shown at 12 that can be adjusted for a particular patient's anatomical proportions as needed. The platform assembly 12 can be ergonomically conformed to different aspects of the patient's body as needed; for example, the patient's height, upper arm length, and forearm reach. The platform assembly includes an exercise step 14, which can be easily adjusted by the patient to greater or lower elevations relative to the platform 12 as needed during the course of the prescribed exercise regime. Such ergonomic adjustments can provide the maximum biomechanical advantage during the exercise session as well as provide the user with a sense of greater stability and security. For patients who are recovering from an injury and may be inclined to avoid exercise out of concern for falling and re-injuring themselves, the ergonomically adjustable platform with adjustable forearm rests 16 and adjustable hand grips 18 can be of great benefit in providing a level of safety and confidence that will promote the frequent and regular use of the system 10. Further, the platform assembly 12 can be provided with an ergonomic taper of the sidewalls, the left side and right side assemblies 56, 58. As shown in FIG. 3, this taper opens the platform assembly and allows the patient's shoulders to rotate laterally (to unlock) as the patient causes his upper body to lower between the sidewalls. Advantageously, this results in increased biomechanical pressure on the knee during flexion.

Also included in the system 10 is a biofeedback assembly, generally shown at 20, that can provide real time biofeedback to the patient during the course of the exercise session. The biofeedback assembly 20 can be useful to the progress of the therapy by providing instantaneous feedback such that the user is aware of his level of success in bending or straightening the post-operative knee and being made so aware can, within self-imposed stiffness and pain limitations, increase his effort to maximize the benefit of each therapy session. Further, the biofeedback assembly 20 can be programmed to include a standardized or personalized ideal course of progress for flexion and extension of the joint so as to provide a running goal for personal achievement by which the patient can measure his daily progress. The embodiment of the biofeedback assembly 20 described herein and shown in the figures is exemplary of the concept of the biofeedback assembly 20, which is only limited by the claims attached hereto. The exemplary biofeedback assembly 20 can include a data collection device exemplified herein as a linear cable encoder 22 in combination with a computing device 24, a data display device 26, and a parameter data input terminal 28. The biofeedback assembly 20 can also include a data transmission device 30 having components, which can be a separate and distinct component or can be at least partially contained within the housing of any of the other components of the biofeedback assembly 20. The system 10 can be battery powered and/or be solely dependent upon connection to an external power source through the power input 32.

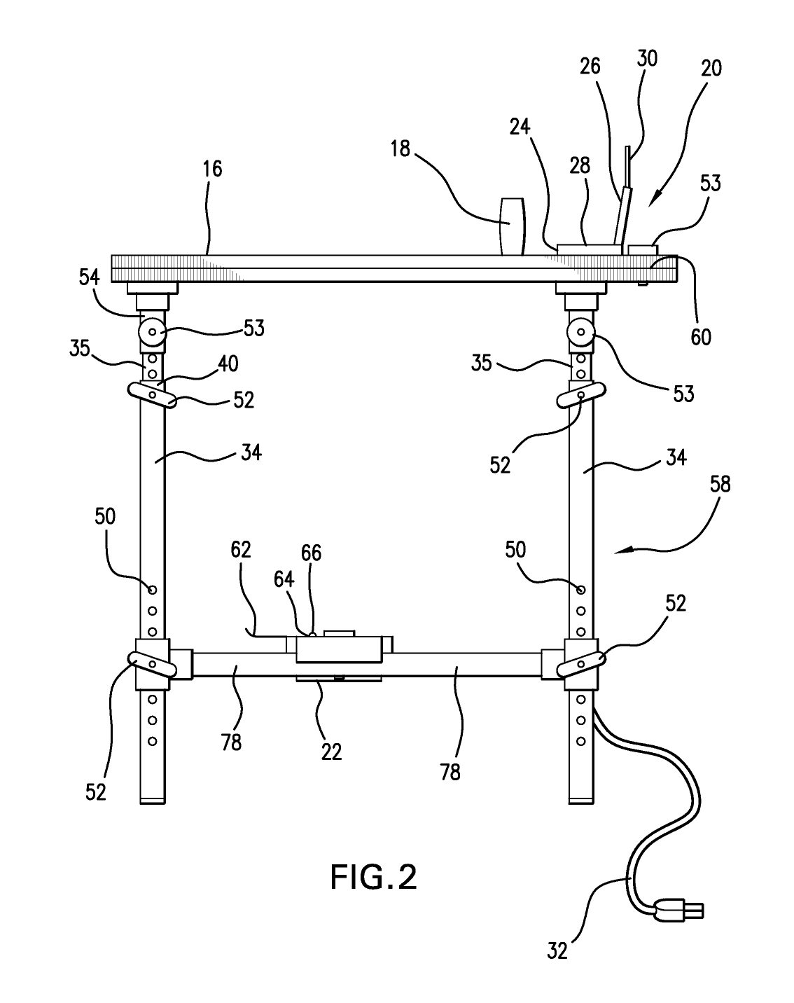

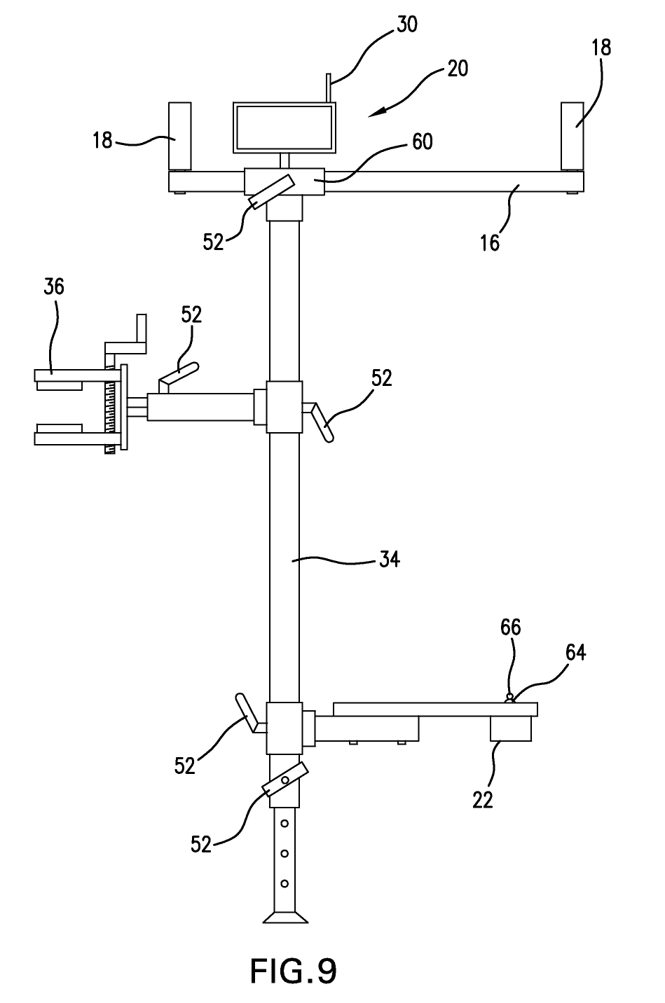

As best shown in FIGS. 1-2 the platform assembly 12 can include at least one upright support 34. It is preferred that the platform assembly 12 include four upright supports; however, it is within the concept of the invention to provide an alternative, but still stable, platform assembly that can have as few as one upright support, as shown in FIG. 9. Providing stability for an embodiment of the present invention that includes less than four upright supports, such as a tripod, a bipod, or a single upright support, might require that the platform assembly be securely connected to a table or counter top, or some other stabilizing structure by a security bracket 36. Preferably, the security bracket 36 can be configured to universally attach to a wide variety of stabilizing structures and be so attached with an easily operated locking and unlocking connection mechanism 38.

The at least one upright support 34, can be preferably provided with a length adjustment mechanism 40 as a means to enable the patient to easily adjust the height of the platform assembly 12 to be ergonomically configured to the patients' body height. Preferably, coordinated adjustment of the upright support length adjustment mechanism 40 for each of the upright supports 34 can be done so as to bring the forearm rests 16, which serve as upper attachments for at least two upright supports 34, to a level that is suitably comfortable for the patient. While any known mechanism for providing an upright support length adjustment feature for the device can be used within the concept of the invention, as shown in FIGS. 1, 2, 4, 5A, and 9, a preferred easily operated length adjustment mechanism 40 can include the well known concept of providing upright support members 34 having internally disposed, extendable telescoping members 35 with a locking mechanism, which can include through holes in the upper portion of the telescoping member 35 and position locking pins, such as for example, quick release ball lock pins 52 sized for easy passage through the locking holes 50. The ball pins 52 conventionally have a push button lock release and are commercially available through companies such as Fairlane Products, Inc.™. While such ball pins are a preferred element of the length adjustment mechanism 40, it is within the concept of the present invention to employ any releasable locking device, such as cotter pins and the like to facilitate the operation of the length adjustment mechanism 40. As best shown in FIG. 1, a left or right pair of upright supports 34 are connected one to the other at their respective tops by the forearm rest 16 and also connected one to the other at a lower position along the shaft of the upright supports by a step support member 78. This additional lower positioned step support member 78 provides a support connection for the exercise step 14 and also provides greater stability to the entire platform assembly.

An additional ergonomic adjustment feature of the present invention that can be provided is an adjustable position hand grip mechanism 42, as shown in FIGS. 1, 2, 3, 4, 5A, and 8. A preferred embodiment of adjustable hand grip mechanism 42, includes two opposing hand grips 18, having hand grip guide members 44 respectively positioned to easily slide within hand grip adjustment slots 46, which are defined along the longitudinal axis and through the body of the forearm rests 16, and hand grip releasable locking retainers 48. Prior to beginning an exercise session, the patient can easily slide the hand grips 18 along the pathway defined by the adjustment slots 46 to a point that is perceived by the patient to be ergonomically suitable. The locking retainers 48 can then be secured so as to hold the hand grips 18 in the selected position for the exercise session. As needed, the patient can easily release the locking retainers 48 and adjust the position of the hand grips 18.

As best shown in FIGS. 1, 2, 4, and 5A, the stability of the platform assembly is derived from the secure connection of the upright supports 34 to the forearm rests 16. Similar to the exemplary telescoping upright length adjustment mechanism 40, this connection of the upright supports 34 to the forearm rests 16 can be easily and securely accomplished by connection of the telescoping members 35 to arm rest connection elements 54 using securing pins, which preferably can be quick release ball lock pins 52, which are sized for easy passage through the locking holes 50 and the armrest connection elements 54. While any other known connection means, such as cotter pins, nut and bolt, screw, and the like can be employed to connect the forearm rests 16 and the upright supports 34, this preferred embodiment provides a secure means of connection that can be easily assembled or disassembled as needed.

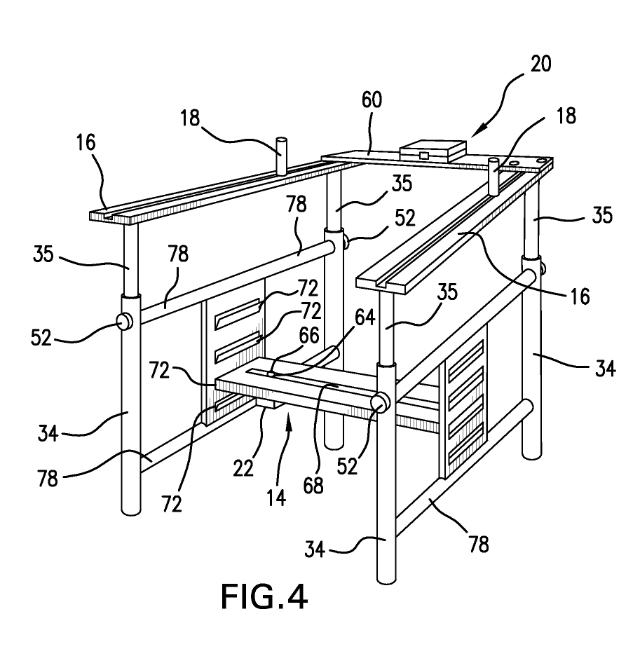

As best shown in FIGS. 1 and 4, in the preferred embodiment, a left side assembly, generally shown at 56, is assembled by the combination of the two upright supports 34 that are connected by the forearm rest 16 that would normally be provided for the patient's left forearm. Similarly, the two upright supports 34 connected to the forearm rest normally provided for the patient's right forearm, when assembled, form a right side assembly, generally shown at 58. A console connection member 60 can be provided, as shown in FIGS. 1, 3, 4, 5A, and 8, to connect the left side assembly 56 to the right side assembly 58. To maximize the strength and stability of the connection of the console connection member 60 to the left and right side assemblies 56, 58, it is preferred that a secure assembly connector 53 be employed such as, for example a threaded bolt engaging a threaded hole defined in the platform or alternatively engaging a threaded nut. For ease of assembly and disassembly of the exercise platform 12, the threaded bolt can be provided with a hand tightened grip such as a fluted knob. Although the strength of such a threaded connection for the console connection member 60 to the upright supports 34 via the interposed telescoping members 35 is preferred, it is within the concept of the invention to provide any known connector for that purpose such as, for example, through holes provided in the components and quick release locking pins 52, cotter pins, or the like.

A final component of the system which is essential to the success of a progressive exercise regime and also adds stability to the platform assembly 12 is the exercise step 14. As shown in FIGS. 1 and 2, the level of the exercise step can be elevated by a simple adjustment of the position of the step 14 using the locking pins 52. While the step 14 does provide greater stability for the platform assembly 12 as a lower connection point between the left side assembly 56 and the right side assembly 58, its primary value to the system is in providing a weight bearing step upon which the patient can place his foot and, as he can bear, press his body weight on to the step and thus exercise the post-operative knee joint. By adjusting the height of the step 14, the patient can lower his upper body weight on to the post-operative knee in a controlled fashion. As the patient progresses in his therapy over time, he can raise the step thereby allowing greater force of body weight in flexing his post-operative or post-injury knee joint. At the lowest step, which appropriately would be used in the earlier part of the rehabilitation program, a mild force would be applied as would be suitable for the pain and discomfort in a recent post-operative knee. As the step is raised to higher levels, the force placed on the post-operative knee would increase. Importantly, the patient using this system can, as his joint stiffness and pain threshold permits, adjust the level of the step to suit his needs without having to rely solely on the judgment of external observations by a therapist.

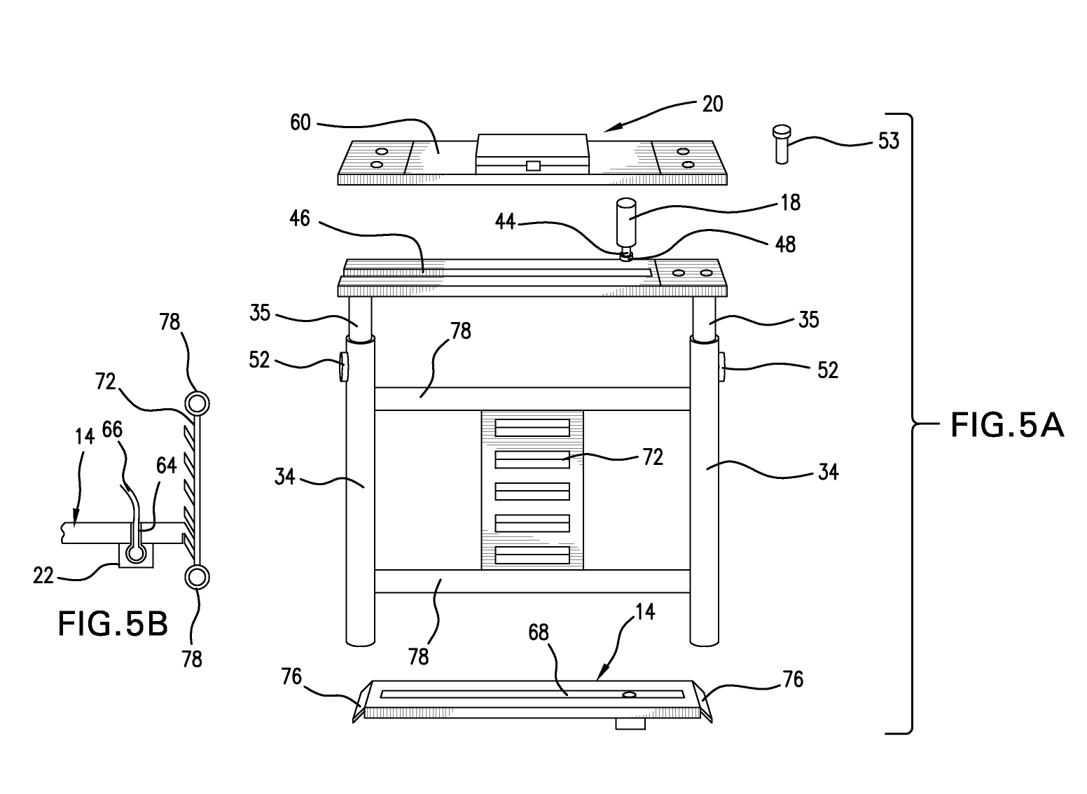

FIGS. 4 and 5 show an alternative mechanism for height adjustment of the step 14. In this alternative embodiment, the height of the step can be adjusted by simply moving the step 14 out of the lateral support grooves 72 located on the step support panel 74, relocating the step 14 to another level, and placing the step back into a secure position. Lateral step supports 76, which are complimentary to the lateral support grooves 72, can be provided on the outer edges of the step 14.

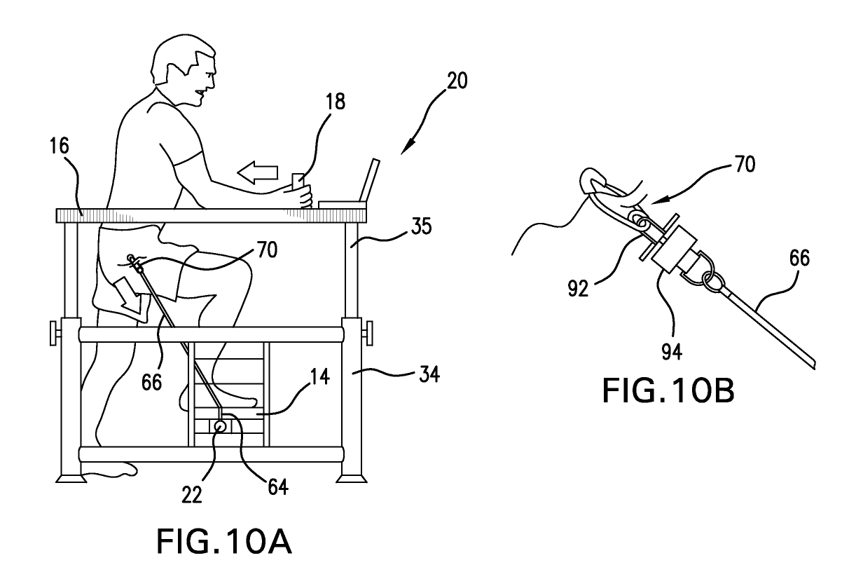

The step 14 can be provided with a foot placement locator 62, which can be a distinct heel locator or guide as shown in FIGS. 1, 2, and 9, or can be simply a position on the step 14 that is adjacent to the location of the linear cable encoder 22. In either case, the foot is properly positioned adjacent to the at least one cable outlet 64 of the linear cable encoder 22. As shown in FIG. 1, the linear cable encoder 22 can be provided with a cable outlet 64 located on each side of the foot placement locator 62. Preferably, a single cable outlet 64, as shown in FIG. 4 can be provided. Importantly, whichever embodiment of the invention is employed, it is important that prior to use the cable outlet 64 be positioned so as to allow extension of the cable 66 on the lateral side of the foot so as to permit extension of the cable upward along the lateral surface of the leg to a point adjacent to the hip joint of the patient. The greater trochanter of the femur is a reproducible prominence on the side of the hip joint, which can be easily located by tactile means. The cable, once extended can be attached to the outer clothing of the patient at a reproducible position over the location of the greater trochanter using any fastener that is suitable to provide a secure releasable attachment. Any variety of pins, clips, hook and loop fasteners, and the like can be used without departing from the spirit of the invention. As shown in FIGS. 10A-B, a preferred method of easily and consistently making the attachment of the cable 66 to the correct anatomical position adjacent the patient's greater trochanter is to provide a separate fastener or cable clip 70 that is configured for easy connection and disconnection to the end of the cable 66. A preferred embodiment is to provide the cable clip 70 as a two-part quick disconnect device as is known in the art. However, this separate clip can be as simple as a safety pin connected to a cable attachment ring, so long as the clip can be easily attached to the outer garment of the patient and when using the device, the patient can easily attach the end of the cable 66 to the clip. As shown in FIGS. 1, 3, 4, 5A, and 8, the linear cable encoder can be slidably positioned to any position selected by the patient along the longitudinal axis of the step 14. The cable outlet 64, in the preferred embodiment extends from the linear cable encoder 22 located beneath the exercise step 14 through guide slot 68 defined along the longitudinal axis of the body of the step 14. In addition to providing the exit portal for the tensioned, coiled cable 66 from the encoder 22, the cable outlet 64 also serves as a guide pin for this sliding movement of the linear cable encoder 22 as the cable outlet 64 travels along the guide slot 68. By this adjustable position feature of the linear cable encoder 22, the patient can selectively position the encoder 22 to a location on the step 14 that is suitable for a therapy program for the left knee or for the right knee as necessary.

In practice, during the initial assembly of the system the patient can adjust the length of the upright supports 34 as well as the position of the hand gripsl 8 to suit his anatomical needs. Such adjustments can be facilitated by pre-marked settings on the upright supports 34 and the forearm rests 16. He positions the step 14 to the proper height adjustment using the preferred adjustment mechanism or the alternative step adjustment mechanism displayed in FIGS. 4 and 5. He can then adjust the position of the linear cable encoder to conform to the left knee or right knee therapy regime as prescribed. The patient then fastens the cable 66 via the cable clip 70 to his outer garment at a position on the lateral side of his leg adjacent to the greater trochanter. As earlier discussed and as best shown in FIG. 10B, this attachment of the cable 66 to the outer garment of the patient is preferably accomplished using a two-part quick disconnect device as the cable clip 70. The patient can attach the upper portion 92 of a quick disconnect clip to the garment and then subsequently attach the cable 66 via the lower portion 94 of the quick disconnect clip at any time throughout the day as the patient desires to use the device 10. When the patient places his foot on the step 14 he is ready to commence the exercise session.

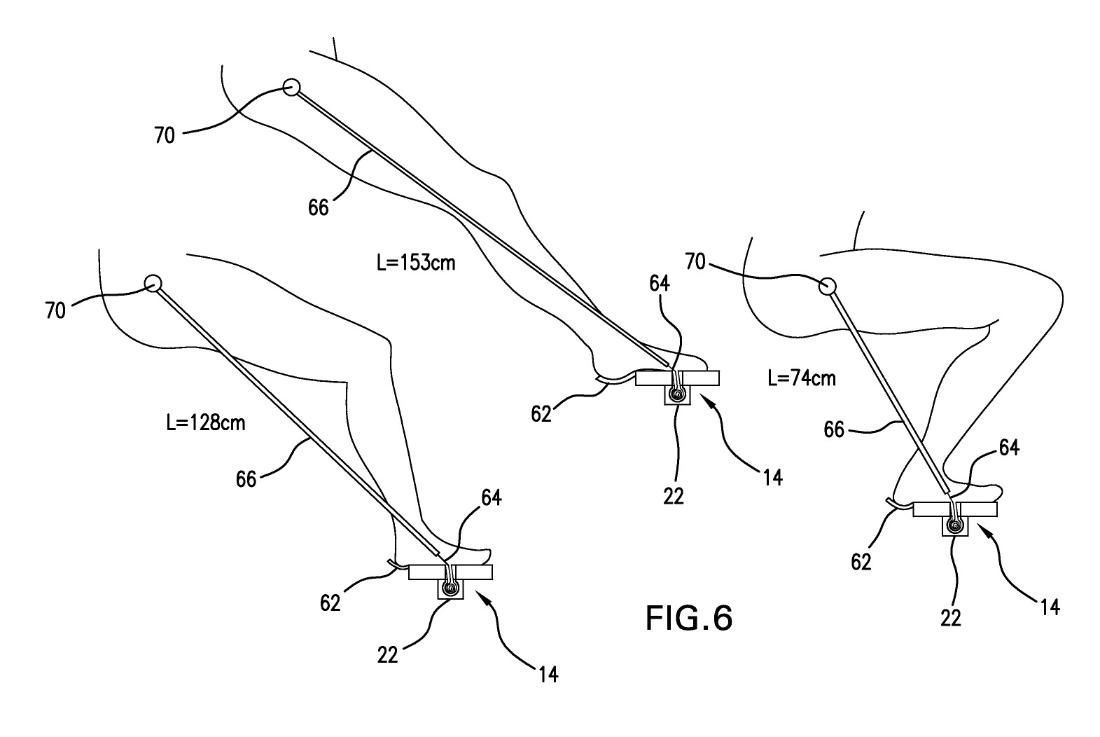

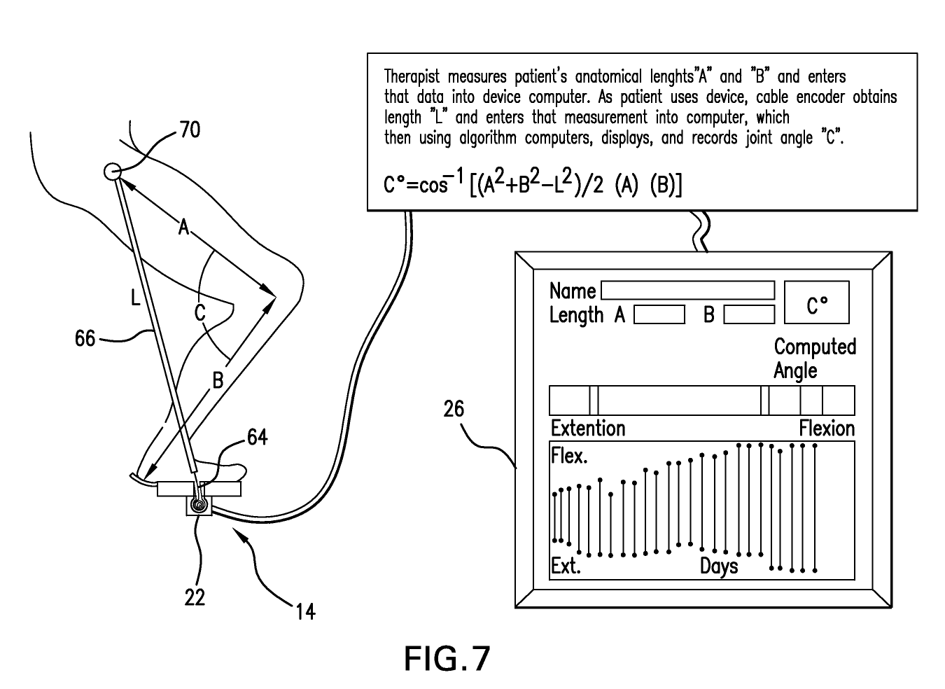

The linear cable encoder 22 of the device 10, in its simplest form, can be provided with a simple counter, which provides a record of the sum of repetitions of the extension and retraction of the cable 66 from the cable encoder 22. As earlier discussed and as shown in FIGS. 6 and 7, it is preferred that the linear cable encoder 22 record the length “L” of the extension of the cable 66 and provide a computation of the angle of the knee during extension and flexion. Prior to beginning exercise repetitions on the device 10 having the preferred embodiment of the cable encoder 22, the patient can note the number on the display for “L” (length of the cable extended by the linear cable encoder) as a comfortable resting position with his foot on the step. An example of a comfortable resting position is 128 mm as shown in FIG. 6. Also shown are exemplary measurements of “L” for extension and flexion. The patient's goal is to make the distance as small as possible while working on flexion of the joint. This is accomplished by lowering his buttocks downward toward his ankle. The cable extends from the cable outlet 64 up to the hip as the patient puts his weight on the step and raises his buttocks up and away from his ankle. The absolute number recorded for “L” is unimportant; it is only important that the patient makes progress in decreasing the number measured as he further flexes his knee. Similarly, in extension, the patient's goal is to increase the number of the measurement for “L”; that is increasing the distance from the buttocks to the ankle. By consistency in positioning of the foot on the step 14 and attachment of the cable clip 70 on the patient's outer garment, the measurement reading obtained by the linear cable encoder 22 will provide a very accurate assessment of the patient's progress. As shown in FIG. 6, the actual measurement of “L” is from the cable outlet 64 of the cable encoder 22, not from the patient's ankle; however, this slight difference in length is not sufficient to be of significance to the computations provided by the biofeedback assembly 20 and further are of no relevance to the goal of the device 10 of providing immediate information of the patient's progress in achieving improved flexion and extension of the knee.

As shown in FIG. 6, the differences in length “L” of the extended cable 66 from the linear cable encoder can vary considerably depending upon the degree of flexion or extension of the knee. The patient's awareness of the differences in these measurements of “L” alone can provide sufficient biofeedback to allow the patient to determine the progression of his therapy program. However, as earlier indicated, and as shown in FIG. 7, the biofeedback assembly 20 of the system 10 in addition to the data collection device exemplified as a linear cable encoder 22 with a simple counter can include a computing device 24, a data display device, a data input terminal, and a data transmission device. The computing device can be programmed with an algorithm for determining the number of degrees in a selected angle of a triangle when the lengths of the three sides of the triangle are known. That algorithm, C°=cos⁻¹[(A²+B²−L²)/2(A)(B)], as shown in FIG. 7, when programmed into the biofeedback assembly 20 of the system 10 will convert the entered measurement data of the anatomical lengths of the upper leg (A) the lower leg (B), and the measured length “L” of the cable of the linear cable encoder 22 into a computed angle (C°) of extension or flexion of the knee. This “angle measurement,” as universally referred to by surgeons and therapists can be displayed immediately to the patient during the course of the exercise session. Such real time biofeedback has never before been possible. In addition to this immediate biofeedback to the patient, the data can be saved for later evaluation by the patient's surgeon or therapist. It is also within the concept of the invention that the data, before or after analysis, be transmitted by cable, satellite, or wireless means to the office of the surgeon and/or therapist to provide a more immediate report on the progress of the patient's prescribed therapy. The frequency of this data link reporting can be programmed for download or transmission on call when the system is prompted by a remote telephonic or computer link prompt from the a requesting party such as the surgeon or therapist or it can be programmed for data transmission at specific times during the prescribed therapy program.

Alternatively, other distance measuring or range finding systems can be employed to determine the degree of movement of a patient's limbs when using the device. Other preferred measurement systems could be used such as, for example, a laser emitter/receiver system attached to the step 14 in place of the most preferred linear cable encoder 22 can be employed to measure the distance from the step 14 to a laser reflector attached to the patient's outer garment adjacent to the hip joint at the position of the greater trochanter without departing from the concept of the invention herein disclosed.

In addition to the benefits of the system discussed above, the platform assembly 12 can be useful for other exercises that are beneficial as the rehabilitation process proceeds. For example, if the patient moves the handle grips 18 back along the forearm rests 16, they can be used as a stabilizing force for exercises of the hip. Toe raises can also be performed using this configuration of the platform assembly 12. Further, if the patient moves the hand grips 18 to their most forward position on the forearm rests 16 and then he steps up onto the step 14 and then down again while maintaining his hold on the hand grips 18, the platform 12 can be employed as a stair stepper to increase quadriceps strength.

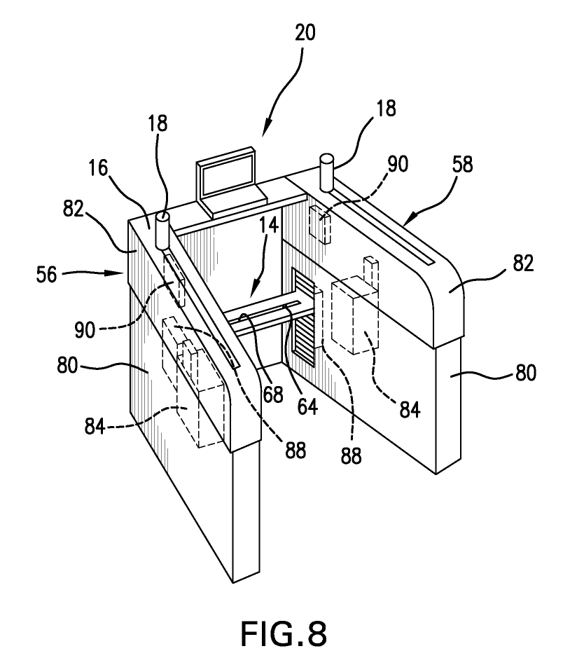

In addition to the exemplary alternative embodiments of the platform assembly 12, which are shown in FIGS. 3, 4, 5A, and 9, the system can be provided with a uni-body component construction platform assembly, such as shown in FIG. 8. In such an embodiment, the strength of the platform can be derived from the entirety of the uni-body construction rather than from the sum of the strength provided by the pipe components of the platform, as shown in FIG. 1. The height adjustment feature for this alternative uni-body construction embodiment can be provided by telescoping left and right side assemblies 56, 58 having a lower portion 80 and an upper portion 82. The upper portion 82 can be slidably displaced upward or downward relative to the lower portion 80 as a means of adjusting the height of the alternative uni-body platform. Servo-assist motors with height position adjustment mechanisms 84, as are well known in the art, can be provided internal to the uni-body construction and are therefore not openly shown in FIG. 8. Additionally, handle servos and adjustment mechanisms 86 and step servos and adjustment mechanisms 88 can be provided to facilitate ergonomic adjustment of these components. This uni-body construction embodiment of the present invention is well suited for a more stationary environment such as the a physical therapy department or a physical therapists office and as such can be equipped with alternative biofeedback systems such as, for example, the well known electromagnetic tracking device known in the art as “Flock of Birds.” Other alternative biofeedback devices can also be employed such as, for example, laser emitter/receiver devices, visual spatial orientation devices, radio-tracking devices, or any other advanced means of determining the angle “C” of a skeletal joint.

The preferred tubular or pipe component construction, as shown in FIGS. 1, 2, 3, 4, 5A-B, 9, 10A, and 11, requires considerable structural strength in each component such as can be provided by aluminum, steel, titanium, or alloys thereof. However, it is within the concept of the present invention that the exercise platform 12 can also be fabricated by wood, high density polymers, light weight composites or any other material having sufficient strength to provide a structurally safe platform assembly 12 for the device 10.

As described above and partially shown in FIG. 5A, the system 10 can be provided as a fully or partially disassembled kit. The kit can be shipped in a container 90 and easily assembled by the patient without need of professional assistance.

Each of the embodiments described above are provided for illustrative purposes only and it is within the concept of the present invention to include modifications and varying configurations without departing from the scope of the invention that is limited only by the claims included herewith.

What is claimed is:

1. A novel exercise therapy system comprising:
a platform assembly, said platform assembly comprising:
at least one elongated upright support, said upright support having a longitudinal axis and an upper end, a middle portion and a lower end, said lower end being configured for stable contact with a supporting surface;
a step, said step being adjustably connected to said at least one upright support and being selectively adjustable upward or downward along said longitudinal axis of said at least one upright support;
a left armrest and a right armrest connected one to the other by a console connection member to form an armrest assembly, said armrest assembly being connected to said upper end of said at least one upright support.

2. The system of claim 1, further comprising a releasable locking mechanism capable of holding said step in a selected position on said at least one upright support.

3. The system of claim 1, wherein at least one of the group of said platform assembly components consisting of at least one upright support, armrests, and step is capable of being selectively adjusted in position relative to said platform assembly.

4. The system of claim 1, wherein said at least one upright support comprises four upright supports disposed one to the other within said exercise platform as two left upright support members forming, in combination with said left arm rest, a left side assembly and two right upright support members forming, in combination with a right arm rest, a right side assembly.

5. The system of claim 4, further comprising:
a left step support member and a right step support member, each of said step support members being configured to adjustably connect the lower portions of the two upright support members of said left side assembly one to the other and to adjustably connect the lower portions of the two upright support members of said right side assembly one to the other, said left and right step support members being configured to provide support for the said step disposed there between, said step being releasably connected to at each end of said step to said left and right step support members.

6. The system of claim 1, further comprising a left hand grip and a right hand grip, each of said left and right hand grips being adjustably connected to said left and said right arm rest respectively, said hand grips having a releasable hand grip locking retainer.

7. The system of claim 6, wherein said left and right hand grips are capable of being adjusted in position along the longitudinal axis of each of said left and right armrests.

8. The system of claim 7, wherein said left and right armrests each comprise hand grip slots configured to guide said left and right hand grips respectively to a selected position on said left and right armrests.

9. The system of claim 1, further comprising a biofeedback assembly releasable connected to said exercise platform, said biofeedback assembly configured to be capable of capturing data regarding the movement of at least one of a patient's limbs during use of said system.

10. The system of claim 9, wherein said biofeedback assembly comprises at least one data collection device.

11. The system of claim 10, wherein said data collection device is at least one linear cable encoder.

12. The system of claim 11, wherein said at least one linear cable encoder is a single linear cable encoder, said linear cable encoder being adjustably attached to said step, said adjustable attachment being capable of selective movement to the left or to the right along the longitudinal axis of said step.

13. The system of claim 12, wherein said linear cable encoder comprises a tensioned coiled cable, said cable having a free end exiting from said linear cable encoder and being attached to a cable clip, said clip being configured for attachment to an outer garment of a patient using said system.

14. The system of claim 13, wherein said cable clip is a quick disconnect member having a first portion and a second portion, said first portion and said second portion being releasably attached one to the other and said first portion being configured for attachment to the outer garment of a patient using the system and said second portion being attached to the free end of said cable.

15. The system of claim 14, wherein said step further comprises at least one foot placement locator.

16. The system of claim 12, wherein said linear cable encoder can be positioned adjacent said at least one foot placement locator.

17. The system of claim 4, wherein said left side assembly and said right side assembly of said platform assembly are ergonomically configured to have an outward taper relative one to the other.

18. The system of claim 13, wherein said biofeedback assembly further comprises a computing device, said computing device being programmable and configured to receive data from said linear cable encoder and compute programmed data and linear cable encoder data to determine an angle “C” of the skeletal joint formed by the pivotal articulation of a first bone with a second bone according to the following algorithm:
C°=cos⁻¹[(A²+B²−L²)/2(A)(B)], where
A is the pre-measured anatomical length of the first bone and
B is the pre-measured anatomical length of the second bone.

19. The system of claim 18, wherein said skeletal joint is the knee joint, said first bone is the femur, and said second bone is tibia.

20. The system of claim 9, wherein said biofeedback assembly comprises at least one of the devices from the group consisting of a data input terminal, a data storage device, a computing device, a data display device, and a data transmission device.

21. The system of claim 9, wherein said biofeedback assembly includes a data collection device selected from the group consisting of a linear cable encoder, a laser emitter/receiver device, an electromagnetic tracking device, a visual spatial orientation device, and a radio-tracking device.

22. The system of claim 1, wherein said exercise platform is manufactured having a uni-body construction.

23. The system of claim 22, further comprising position adjustable hand grips, armrests, and step, wherein said position adjustment is augmented by power servos.

24. The system of claim 23, wherein said biofeedback assembly includes a data collection device selected from the group consisting of linear cable encoder, a laser emitter/receiver device, an electromagnetic tracking device, a visual spatial orientation device, and a radio-tracking device.

25. The system of claim 1, wherein said exercise platform is constructed of at least one of the materials of the group consisting of aluminum, steel, titanium, high density plastics, polymeric composites, wood, and alloys of aluminum, steel, and titanium.

26. A method of providing joint exercise therapy for a patient in need thereof, the method comprising:
providing the system of claim 1;
determining the length of each of the two skeletal bones that are in pivotal articulation one to the other to form a knee joint angle “C;”
ergonomically adjusting said platform assembly 1 to fit the anatomy of a patient in need of therapy;
positioning said step to a starting position suitable for said patient, said patient placing patient's foot of the side in need of therapy on said step;
patient increasing pressure downward on said step raising body upward relative to said platform assembly so as to bring body weight to bear on said step and causing extension of said knee joint angle “C;”
patient relaxing pressure on said step so as lower body downward relative to said platform and decrease body weight pressure on said step causing flexion of said joint angle “C;”
adjusting step position upward relative to said platform assembly;
repeating increasing pressure step and relaxing pressure step,
whereas said repetitive steps and upward step position adjustments provide therapy for said knee joint and improve knee flexion and extension as reflected in degree changes of knee joint angle “C.”

27. The method of claim 26, wherein said platform assembly further comprises a biofeedback assembly, said biofeedback assembly comprises a data collection device.

28. The method of claim 27, wherein said data collection device is selected from the group consisting of a linear cable encoder, a laser emitter/receiver device, an electromagnetic tracking device, a visual spatial orientation device, and a radio-tracking device.

29. The method of claim 28, wherein said data collection device is a linear cable encoder, said linear cable encoder being attached to said platform assembly step and said method further comprises:
attaching a free end of a tension, coiled cable from said linear cable encoder to an outer garment of said patient at a position adjacent to the greater trochanter of said patient's femur;
patient monitoring of said biofeedback assembly during therapy sessions to determine degree of improvement of flexion and extension of said knee joint.

30. A kit comprising a system according to claim 1 and instructional material for assembly and use of said system.

31. The kit of claim 30, further comprising a container for said kit.
